(19) United States
(12) Patent Application Publication
(10) Pub. No.: US 2006/0114931 A1
(43) Pub. Date: Jun. 1, 2006

(54) IPv6/IPv4 packet conversion system

(75) Inventor: Hiroshi Miyata, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: YOKOGAWA ELECTRIC CORPORATION, Musashino-shi (JP)

(21) Appl. No.: 10/998,568

(22) Filed: Nov. 30, 2004

(51) Int. Cl. H04J 3/22; H04J 3/16; A61K 39/29

(52) U.S. Cl. 370/466

(57) ABSTRACT

An object of the present invention is to achieve an IPv6/IPv4 packet conversion system capable of TCP-compliant packet size control while executing packet conversion between IPv4 and IPv6 networks. Accordingly, the system of the present invention comprises an IPv6/IPv4 translator installed between IPv6 and IPv4 networks and equipped with a means to output MSS data of a specific value to the IPv4 server when the translator performs packet conversion between IPv6 and IPv4 protocols. The MSS output means employs: a) An MSS conversion block for converting MSS data contained in a session initiation packet input by an IPv6 client into MSS data of a specified value and outputting that data to an IPv4 server. b) An MSS check block for checking the existence of MSS data in a session initiation packet input by an IPv6 client and MSS conversion block for, in the case of the absence of MSS data in the session initiation packet, adding MSS data of a specified value to the packet for outputting to the IPv4 server. These features enable packet size control by adjusting the MSS value of the TCP without the issuance of the “Packet Too Big” message of the ICMP.

### BACKGROUND OF THE INVENTION

1. Field of the Invention

The present invention relates to an IPv6/IPv4 packet conversion system and, more specifically, to a system for converting packets in order to stabilize communications between a terminal that employs IPv6 (Internet Protocol version 6) as the communication protocol and another terminal that employs IPv4 (Internet Protocol version 4) also as the communication protocol.

2. Description of the Related Art

IPv6 is a next-generation Internet protocol. The currently used 32-bit IPv4 can offer up to approximately 4.3 billion addresses. However, because of the rapid diffusion of the Internet, the shortage of addresses is becoming a serious problem. The IETF (Internet Engineering Task Force) developed the next-generation Internet protocol, IPv6 which has some features that IPv4 does not have. Also, IPv6 is a 128-bit protocol, meaning that it can accommodate approximately 3.4×10³⁸ addresses.

FIG. 1 illustrates the TCP packets of IPv4 and IPv6. (A) and (B) represent the TCP packets of IPv4 and IPv6, respectively. As FIG. 1 indicates, the IP header size of the IPv4 packet is 20 bytes, and that of the IPv6 packet is 40 bytes. IPv4 offers an option to increase the header size. An IPv6 packet can have an extension header.

An IPv6/IPv4 translator needs to replace an IP header to execute packet conversion. The size of the parts of the TCP packet other than the IP header is not affected by packet conversion. The size of an IPv6 packet converted from an IPv4 packet is larger than the size of the original IPv4 packet by 20 bytes.

Let us assume that when the translator converts an IPv4 packet into an IPv6 packet, the MTU (Maximum Transmission Unit) of the link where the IPv4 packet has reached is 1500 bytes, and the MTU of the link whereto the link that is to transfer IPv6 packets is destined, is 1500 bytes. If the size of the IPv4 packet to be converted is 1500 bytes, the converted packet is 1520 bytes. The MTU of the link that transfers IPv6 packets is 1500 bytes, so a 1520-byte packet cannot be transferred.

The header of an IPv4 packet has a “Don't Fragment (DF)” flag, which indicates whether or not packet fragmentation is allowed. DF=0 indicates that the packet can be fragmented by a router along the transmission path thereof. DF=1 indicates that the packet cannot be fragmented by a router along the transmission path thereof.

FIG. 2 explains how IPv4 packets are processed when DF=0. FIG. 3 explains how such packets are processed when DF=1. IPv6/IPv4 translator 10 comprises is comprised of DF check block 11, Packet fragmentation block 12, and Error message transmission block 13.

When the size of an IPv4 packet is 1500 bytes and DF=0 (1), based on the check results by DF check block 11, Packet fragmentation block 12 fragments the packet to be transferred in the form of IPv6. As FIG. 2 shows, the IPv4 packet is divided into 1496-byte and 100-byte IPv6 packets (2). In contrast, when DF=1 (1) , the packet cannot be fragmented. FIG. 3 depicts that based on the check results by DF check block 11, Error message transmission block 13 transmits to the transmission end an ICMP (Internet Control Message Protocol) error message (Packet Too Big) , which contains a value that specifies an appropriate packet size (2). In this case, the transmission end receives a message saying that 1480 bytes is an appropriate size. Upon receiving this message, the transmission end thereafter adjusts the size of the packet to the specified size. In the case of FIG. 3, a 1480-byte IPv4 packet is transmitted (3). When this 1480-byte IPv4 packet is converted into an IPv6 packet, the size of the converted IPv6 packet is 1500 bytes (4). Translator 10 can transfer this packet without fragmentation.

FIG. 4 presents another method to control the size of data to be transferred at the TCP level using Maximum Segment Size (MSS). As presented in FIG. 4, Client 20 is comprised of MSS request transmission block 21, MSS acknowledgement transmission block 22, Data transfer request block 23, and Data reception block 24. Server 30 is comprised of MSS comparison and transmission block 31, MSS acknowledgement reception block 32, and Data transfer block 33.

In this configuration, data size control is performed in the following procedure:
(1) Client 20 initiates a session. MSS request transmission block 21 of Client 20 notifies Server 30 of the desired MSS.
(2) In response, MSS comparison and transmission block 31 of Server 30 compares the desired segment size with the MSS received from the client and notifies Client 20 of the smallest possible MSS, which is to be used consistently later in the session.
(3) MSS acknowledgement transmission block 22 of Client 20 sends a response to Server 30 (the final step of the 3-way handshake of the TCP).
(4) Data transfer request block 23 of Client 20 requests Server 30 to transfer data.
(5) Data transfer block 33 of Server 30 transfers data to Data reception block 24 of Client 20. The TCP segment size is 1440 bytes, which is the initially adjusted value.

In a communication system that employs the devices shown in FIG. 2 and FIG. 3, when the DF of an IPv4 packet is 1, the transmission end thereof must perform communications according to the specified MTU value. However, in some cases, communication cannot be established because this MTU value is not used. As shown in FIG. 5, the transmission of 1500-byte packets (1) and (3) and the issuance of the “Packet Too Big” messages (2) and (4) are endlessly repeated. Devices whose practical use has been recently achieved offer a forced fragmentation function. Such devices are equipped with Forced packet fragmentation block 14, so that they can forcibly fragment IPv4 packets as necessary regardless of the DF value.

[Patent Document 1]

Japanese Laid-open Patent Application 2000-115234

Paragraph 0049 of Patent document 1 refers to header conversion between IPv6 and IPv4 packets. However, unlike the present invention, it does not address packet size control.

However, in a configuration where Translator 10 fragments packets (see FIG. 5), excessive loads are exerted on the translator.

### SUMMARY OF THE INVENTION

The present invention is aimed at solving the aforementioned problems. An object of the present invention is to achieve an IPv6/IPv4 packet conversion system capable of TCP-compliant packet size control while executing packet conversion between IPv4 and IPv6 networks.

Accordingly, the system of the present invention comprises an IPv6/IPv4 translator installed between IPv6 and IPv4 networks and equipped with a means to output MSS data of a specific value to the IPv4 server when the translator performs packet conversion between IPv6 and IPv4 protocols.

The MSS Output Means Employs:

a) MSS conversion block for converting MSS data contained in a session initiation packet input by an IPv6 client into MSS data of a specified value and outputting that data to the IPv4 server.
b) MSS check block for checking the existence of MSS data in a session initiation packet input by an IPv6 client and MSS conversion block for, in the case of the absence of MSS data in the session initiation packet, adding MSS data of a specified value to the packet for outputting to the IPv4 server.

The present invention enables packet size control by adjusting the MSS value of the TCP without the issuance of the “Packet Too Big” message of the ICMP.

### BRIEF DESCRIPTION OF THE DRAWINGS

FIG. 1 is a diagram illustrating TCP packets.

FIG. 2 is a diagram illustrating IPv4 packet processing when DF=0.

FIG. 3 is a diagram illustrating IPv4 packet processing when DF=1.

FIG. 4 is a diagram illustrating a method to control the size of transferred data at the TCP level.

FIG. 5 is another example of a conventional packet processing system.

FIG. 6 is a conceptual block diagram that represents an example embodiment of the present invention.

FIG. 7 is a conceptual block diagram that represents another example embodiment of the present invention.

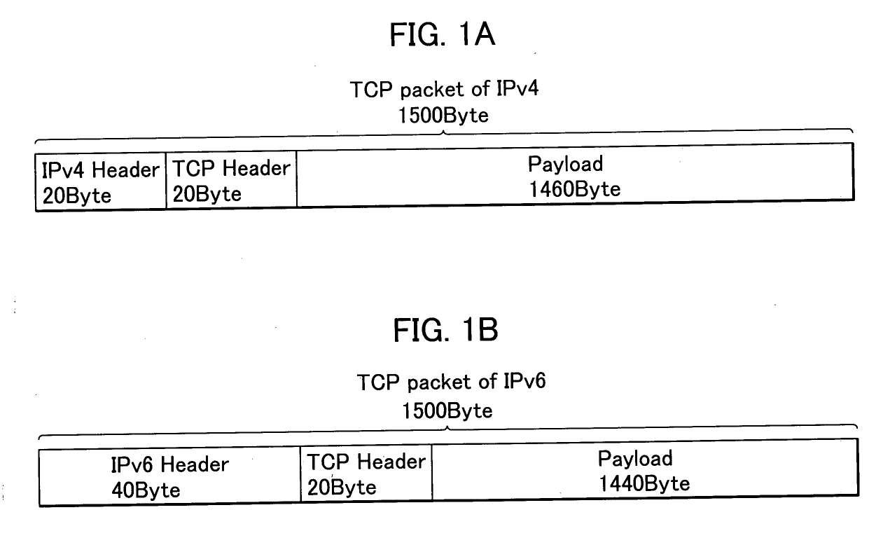

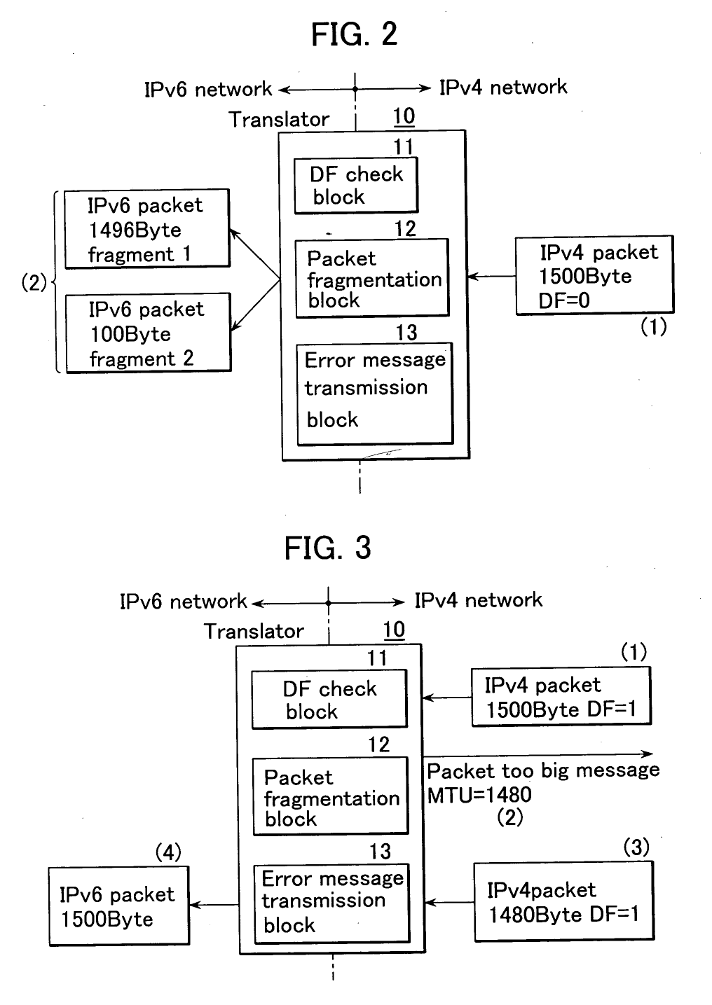

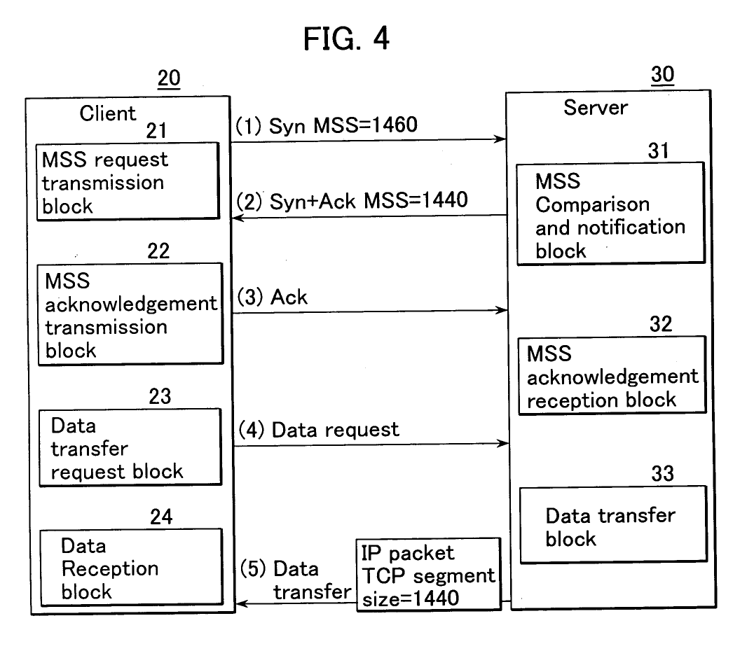

### DESCRIPTION OF THE PREFERRED EMBODIMENTS

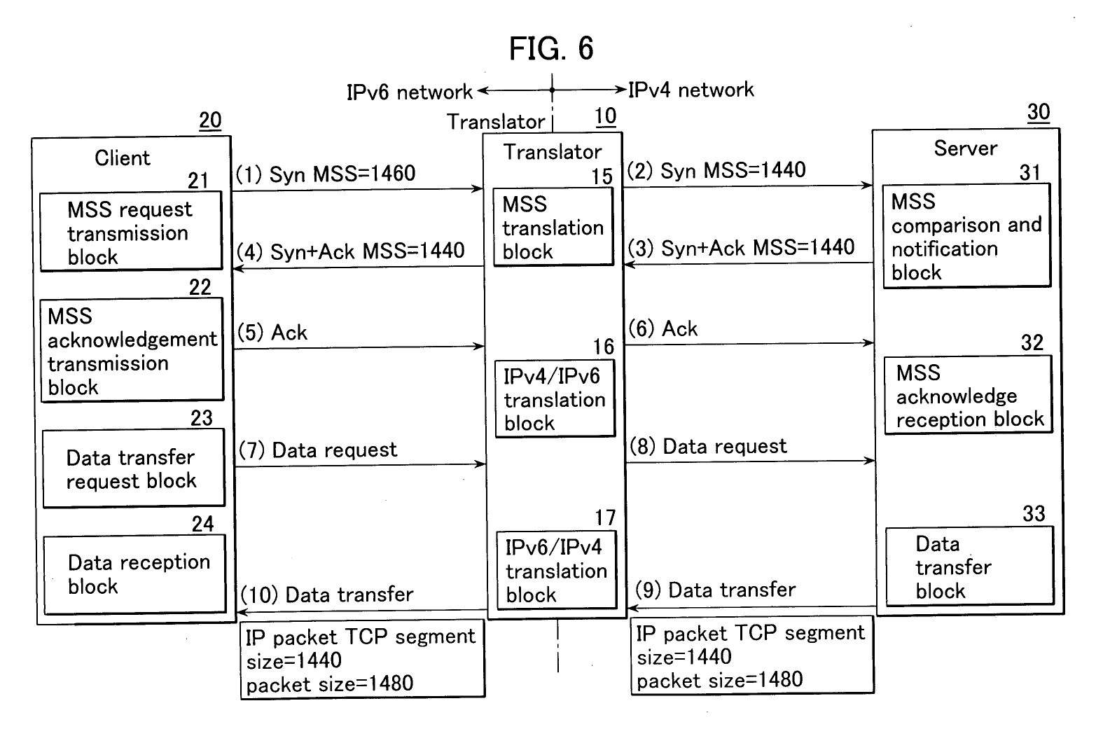

Preferred embodiments of the present invention are described hereinafter in detail by referring to the accompanying drawings, wherein FIG. 6 is a conceptual block diagram illustrating an example embodiment of the present invention. In FIG. 6, IPv6/IPv4 translator 10 is comprised of MSS conversion block 15, which acts as the MSS output means; IPv4/IPv6 conversion block 16, and IPv6/IPv4 conversion block 17. IPv6 client 20 is comprised of MSS request transmission block 21, MSS acknowledgment transmission block 22, Data transfer request block 23, and Data reception block 24. IPv4 server 30 is comprised of MSS comparison and notification block 31, MSS acknowledgment reception block 32, and Data transfer block 33.

The behavior of the embodiment illustrated in FIG. 6 is described below:
(1) IPv6 client 20 initiates a session. MSS request transmission block 21 of Client 20 notifies IPv4 Server 30 of the desired MSS.
(2) MSS conversion block 15 of IPv6/IPv4 translator 10 checks the MSS contained in the session initiation packet input by IPv6 client 20. Then MSS conversion block 15 converts the MSS into an MSS whose value indicates that the converted MSS is smaller than the original by 20 bytes, which is the size difference between the IPv6 and IPv4 headers, before IPv4 server 30 is notified of the converted MSS value.
(3) In response, MSS comparison and notification block 31 of IPv4 server 30 compares the desired segment size with the MSS received from the client and notifies Client 20 of the smallest possible MSS, which is to be used consistently later in the session.
(4) IPv4/IPv6 conversion block 16 of IPv6/IPv4 translator 10 converts the IPv4 packet in (3) above into an IPv6 packet for transmission to IPv6 client 20. The value specified here is smaller than the one specified in (2) above, so the value specified here does not need to be checked.
(5) MSS acknowledgment transmission block 22 of IPv6 client 20 sends a response to Server 30 (the final step of the TCP 3-way handshake).
(6) IPv6/IPv4 conversion block 17 of IPv6/IPv4 translator 10 converts the IPv6 packet in (5) above into an IPv4 packet for transmission to IPv4 server 30.
(7) Data transfer request block 23 of IPv6 client 20 transmits a data transfer request to Server 30.
(8) IPv6/IPv4 conversion block 17 of IPv6/IPv4 translator 10 converts the IPv6 packet in (7) above into an IPv4 packet for transmission to IPv4 server 30.
(9) Data transfer block 33 of IPv4 server 30 transfers data to Client 20. Here, the TCP segment size is 1440 bytes, which is the initially adjusted value. Likewise, the IPv4 packet size is 1480 bytes.
(10) IPv4/IPv6 conversion block 16 of IPv6/IPv4 translator 10 converts the packet in (9) above into an IPv6 packet for transmission to IPv6 client 20. Here, the TCP segment size is 1440 bytes, which is the initially adjusted value. Likewise, the IPv6 packet size is 1500 bytes.

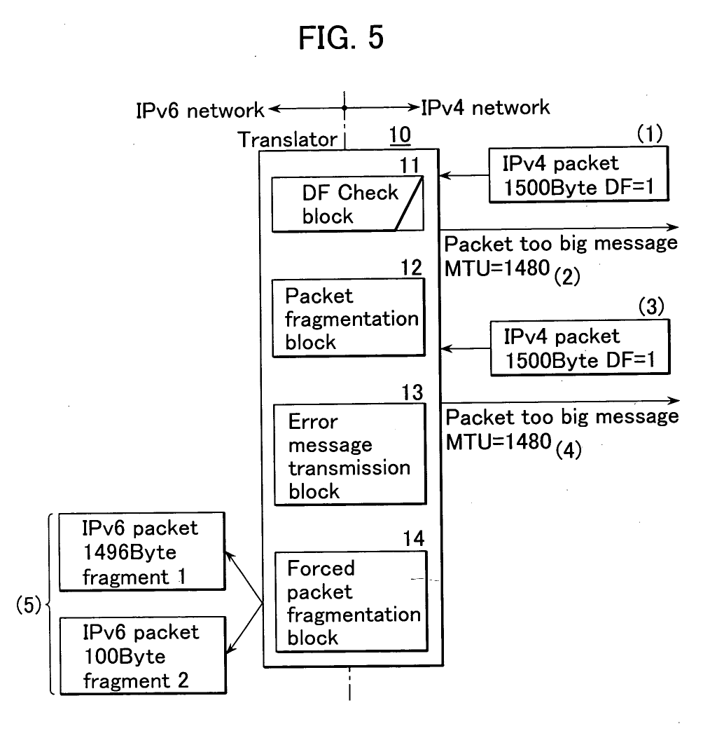

The “Packet Too Big” message, transmitted by Error message transmission block 13 of Translator 10 (see FIG. 3 and FIG. 5), does not always work effectively. The reason is that some ICMP messages do not reach the equipment that transmitted the 1500-byte IPv4 packet because of firewalls, filtering, or the like along the transmission path.

In contrast, the present invention enables packet size control by adjusting the MSS value of the TCP without the issuance of the “Packet Too Big” message of the ICMP.

In this way, packet size control is more likely to work for a host which cannot effectively receive the “Packet Too Big” message. Packets whose size does not require fragmentation can be sent from the transmission end, so the load on IPv6/IPv4 translator 10 can be reduced.

When the load from packet fragmentation is reduced for IPv6/IPv4 translator 10, the throughput of translator 10 is expected to improve.

The retransmission of the “Packet Too Big” message and packets is reduced, and so is network traffic.

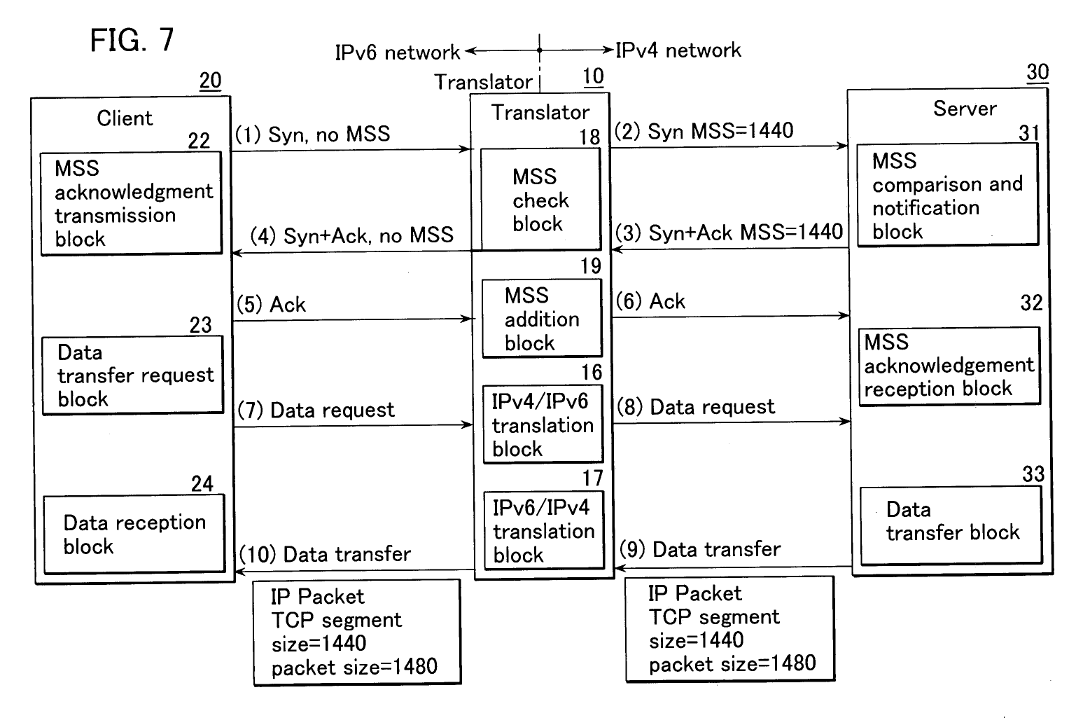

In the TCP, the MSS adjustment function is optional. An MSS may be absent in a session initiation packet from an IPv6 client. In such cases, as shown in FIG. 7, MSS check block 18 and MSS addition block 19 should be added to IPv6/IPv4 translator 10, so that they act as an MSS output means. When IPv6/IPv4 translator 10 performs conversion for a packet without an MSS, sending an IPv4 packet with an MSS option will achieve similar results to those of the aforementioned MSS value adjustment.
(1) IPv6 client 20 initiates a session. Now assume that the session initiation packet does not have an MSS.
(2) MSS check block 18 of IPv6/IPv4 translator 10 checks whether MSS data exists in the packet initiation session. If it does not exist, MSS addition block 19 uses an MSS to notify IPv4 server 30 of the size of the packet that does not require conversion when IPv6/IPv4 conversion block 17 converts an IPv4 packet into an IPv6 packet. Specifically, when the MTU of the IPv6 packet is 1500 bytes, the server is notified of 1440 bytes, which is equal to 1500 bytes minus the combined size of the IPv6 header (40 bytes) and the TCP header (20 bytes).
(3) In response, MSS comparison and notification block 31 of IPv4 client 30 compares the desired segment size with the MSS received from the client and notifies IPv6 client 20 of the smallest possible MSS, which is to be used consistently later in the session.
(4) IPv4/IPv6 conversion block 16 of IPv6/IPv4 translator 10 converts the IPv4 packet in (3) above into an IPv6 packet for transmission to IPv6 client 20. The value specified here is smaller than the one specified in (2) above, so the value specified here does not need to be checked.
(5) MSS acknowledgment transmission block 22 of IPv6 client 20 sends a response to Server 30 (the final step of the TCP 3-way handshake).
(6) IPv6/IPv4 conversion block 17 of IPv6/IPv4 translator 10 converts the IPv6 packet in (5) above into an IPv4 packet for transmission to IPv4 server 30.
(7) Data transfer request block 23 of IPv6 client 20 transmits a data transfer request to Server 30.
(8) IPv6/IPv4 conversion block 17 of IPv6/IPv4 translator 10 converts the IPv6 packet in (7) above into an IPv4 packet for transmission to IPv4 server 30.
(9) Data transfer block 33 of IPv4 server 30 transfers data to Client 20. Here, the TCP segment size is 1440 bytes, which is the initially adjusted value. Likewise, the IPv4 packet size is 1480 bytes.
(10) IPv4/IPv6 conversion block 16 of IPv6/IPv4 translator 10 converts the packet in (9) above into an IPv6 packet for transmission to IPv6 client 20. Here, the TCP segment size is 1440 bytes, which is the initially adjusted value. Likewise, the IPv6 packet size is 1500 bytes.

The IPv6/IPv4 packet conversion system according to the present invention enables packet size control by adjusting the MSS value of the TCP without the issuance of the “Packet Too Big” message of the ICMP. The load on the IPv6/IPv4 translator is reduced, so the throughput is expected to improve. The retransmission of the “Packet Too Big” message and packets is reduced, and so is network traffic. The present invention affords many benefits in a practical sense.

What is claimed is:

1. An IPv6/IPv4 packet conversion system, wherein an IPv6/IPv4 translator installed between an IPv6 network and an IPv4 network executes packet conversion therebetween, wherein said IPv6/IPv4 translator is equipped with an MSS (Maximum Segment Size) output means whereby MSS data of a specified value is output to an IPv4 server.

2. The IPv6/IPv4 packet conversion system in accordance with claim 1, wherein said MSS (Maximum Segment Size) output means employs an MSS conversion block to convert MSS data contained in a session initiation packet input by an IPv6 client into MSS data of a specified value for transmission to said IPv4 server.

3. The IPv6/IPv4 packet conversion system in accordance with claim 1, wherein said MSS (Maximum Segment Size) output means employs an MSS check block to check the presence of MSS data in a session initiation packet input by an IPv6 client and, if said MSS data is not present in said session initiation packet, uses an MSS conversion block to add MSS data of a specified value to said session initiation packet for transmission to said IPv4 server.
